(19) United States
(12) Patent Application Publication
(10) Pub. No.: US 2013/0131407 A1
(43) Pub. Date: May 23, 2013

(54) CATALYTIC HYDROGENATION OF HYDROXYCYCLOALKANES AND USE OF THE PRODUCT IN BIOFUEL COMPOSITIONS FOR AVIATION

(71) Applicants: Marco Andre Fraga, Rio Janeiro (BR); Luis Eduardo Pizzaro Borges, Rio Janerio (BR); Flavio Dos Reis Goncalves, Rio Janerio (BR)

(72) Inventors: Marco Andre Fraga, Rio Janeiro (BR); Luis Eduardo Pizzaro Borges, Rio Janerio (BR); Flavio Dos Reis Goncalves, Rio Janerio (BR)

(21) Appl. No.: 13/812,755

(22) PCT Filed: Jul. 28, 2011
(86) PCT No.: PCT/BR11/00248

§ 371 Date: Jan. 28, 2013

(30) Foreign Application Priority Data
Jul. 29, 2010 (BR) PI1003516-8

(51) Int. Cl. C10L 1/04; C07C 1/20

(52) U.S. Cl. CPC C10L 1/04; C07C 1/20
USPC 585/14; 585/357

(57) ABSTRACT

This invention relates to a new biofuel alternative to be used in aviation sector, starting from obtention and production routes of renewable sources compounds, that may act as load for aviation kerosene composition. Naphthenic compounds (cycloalkanes) obtained from renewable sources are used as enrichment or addition loads of aviation kerosene. The process is based on hydrogenolysis catalytic reactions, from hydroxycycloalkanes derevatives substrata, like menthol and isopulegol. The catalytic system is constituted of a physical mixture of hydrogenation heterogeneous catalysts, acid heterogeneous catalysts, and hydrogenating metallic catalysts in acid supports. The hydrogenation catalysts used envolve noble metals from groups 6, 7, 8, 9 and 10 of periodic table, whose content ranges from 0.01-10%. The heterogeneous catalysts suitable acids are represented by acidic sulfonated polymer resins, protonated zeolites and sulfated zirconia. The catalytic reaction conditions involving a temperature range of 70-250° C., pressure between 1-70 and agitation ranging from 100-1000 rpm. The composition involving the biofuel, obtained by catalytic hydrogenation process, is obtained as a mixture composed by cycloalkanes and aviation fuel in ratio 1:100 to 100:1, in volume.

### CROSS REFERENCE TO RELATED APPLICATION

This application is a national stage entry of PCT/BR2011/000248 filed Jul. 28, 2011, under the International Convention claiming priority over Brazilian Patent Application No. P11003516-8 filed Jul. 29, 2010.

### FIELD OF THE INVENTION

This invention intends to assess catalytic processes in single phase from hydroxycycloalkanes based substrata to obtain, mainly, cycloalkanes derivatives, among which compounds like p-menthane and 1-isobutyl 3-methyl cyclopentane and their use in aviation fuel formulation, through renewable material as start substrata, as is the case with menthol. The possibility of insertion of cycloalkanes to aviation kerosene is due to the low freezing point, −118° C. (source: http://webbook.nist.gov/chemistry/name-ser.htlm), resistance to oxidation and absence of sulfur and nitrogen in these compounds molecular structure. Such factors are advantageous because of fossil components reduction in aviation fuels composition, especially from aromatic components, which are responsible for soot or particulates formation during combustion process, and are carcinogenic substances, therefore, harmful to one's health during fuel handling.

The CO₂ equivalent emissions from fossil fuels in aeronautical sector respond to 2-3% of global emission. In this way, the use of renewable biofuel, with low freezing point, and resistant to oxidation, can partially replace fossil sources in aviation fuels composition and mitigate greenhouse effect gases, without need of significant change in aeronautical engines technology, especially in jet turbine propulsion and turbo engine propulsion airplanes and airplanes that use alternative or combustion engine.

### SUMMARY OF THE INVENTION

This patent application presents new alternatives to biofuel production for use in aviation sector. The air transport segment, only in Brazil, has consumed around 5.2 million of cubic meters of aviation kerosene (AK-1) in 2008 (ANP Statistic Yearbook, 2009), thus generating thousands of tons in emissions of CO₂, NOₓ and other gases formed by combustion. It is worth highlighting that aviation kerosene (AK-1) marketed in Brazil is equivalent and compatible with specifications of AFQRJOS (Aviation Fuel Quality Requirements for Jointly Operated Systems) Jet A-1.

Besides, fossil origin aviation kerosene contains around 20% in aromatic substances volume, and the tendency is to have it replaced due to its carcinogen character.

Jet propulsion airplanes operate under extreme temperature conditions, both externally, where kerosene is stored in storage tanks inserted in airplanes' wings, exposed to temperatures ranging from −50° C. to −70° C., and internally, where turbine structure is heated by exhaustion gases and air friction, reaching temperatures of approximately 482° C. High temperatures occurring in combustion chamber proximity, especially in airframes and turbine subsystems, promote pre-heating of kerosene in feed-lines, which can promote kerosene oxidation due to formation of peroxides, which consequently propitiate the formation of gums.

Hence, molecular components that show low freezing point, good calorific value and structural characteristics that resist to fuel degradation before burning phase of combustion chamber are natural candidates to formulation of mixtures like commercial aviation kerosene. Such features are observed in naphthenic molecular components, particularly in cycloalkanes, which can be used not only in combustible compositions involving AK-1, but in aviation gasoline as well, or in any other aviation fuel.

Another determining factor in the search for alternative sources for total or partial replacement in fossil aviation kerosene comes from the increase of oil consumption due to global economy increase, which pushes oil and derivatives quotation in the international market. Political conflicts involving the Middle East, estimates of world production fall within 35 to 50 years, according to the number of reserves proved and registered, both contribute to scenery with increasing prices of oil and derivatives.

Starting from these facts, it is justified, today, the development of processes that use alternative and renewable sources to minimize current consumption of existing and proved oil reserves, and consequently establish new chemical processes to obtain fuels from renewable sources. At the same time, the effects of fossil fuels burning have caused changes in Earth climate conditions, due to the action of gases that generate greenhouse effect. Environmental pressures have generated a series of volunteer initiatives or initiatives linked to Kyoto Protocol to reduce in 5.2% greenhouse effect gases emissions levels as compared to corresponding emissions in 1990.

Brazil develops for more than 35 years scientific and technological researches in renewable fuels segment which have propitiated, in time, the consolidation of ethanol as alternative fuel to gasoline with more than 50 patents deposited. More recently, in 2005, the Brazilian government has established the National Program for Production and Use of Biodiesel (PNPB), making mandatory, through Law 11097, that diesel distributors offer B2 mixture (addition of 2% of biodiesel to oil diesel) as a first commitment. Today, the addition of biodiesel to diesel, which is offered to consumer at the gas pump, has already gone beyond the 3% value transition barrier, and is an addition that corresponds to B5 mixture, which was supposed to be introduced in market and be available to consumer in 2012.

Brazilian government and scientific community actions have attracted world interest in these energetic as fuel alternative renewable sources, aligned to their expectations of conscious consumption. Large territorial areas, sun energy during the whole year, plant variety technology, renewable raw materials noncompetitive in relation to food and development of injection system to fossil and renewable fuels mixtures, have all contributed to place Brazil in the forefront in the replacement of fossil fuels for renewable sources from biomass.

However, the development of technologies from alternative renewable sources, whose products can be added to aviation kerosene is not well defined yet. According to US Air Force (USAF) studies indicate that until 2020 there will be a strong possibility of not having available alternative sources to aviation kerosene, which could meet supply needs in scale demanded by world economy growth.

The alternative of replacement of up to 50% of naphthenic and aromatic molecules present in representative distillation cuts of aviation kerosene, by simple ring molecules obtained by catalytic cracking of sources like coal, schist or tar is seen as promising by US Air Force (USAF). In few words, this potential replacement can be tested in a sustainable way through the use of cyclic molecules from renewable sources, represented in this application, for example, by p-methane, which may contribute to the development of an aviation fuel formulation with lower aromatic contents (carcinogenic) and sulfur, and may also reduce particulates formation during their combustion process. The safety in fuel handling and reduction of greenhouse effect gases propitiated by content reduction, particularly aromatic, may also promote synergies owing to the smaller amount of aromatics present in the composition, and to the lower amount of fossil in fuel, respectively. On the other hand, fossil aromatics, present in conventional kerosene may be applied in chemical processes to obtain higher added value products mainly through their use in petrochemical processes for plastic, resins, chemical intermediates for medicines, agrochemical and chemical specials production.

### BACKGROUND OF THE INVENTION

The status of technique describes reference WO2009/051462A1, application of menthol in fuel compositions. It reveals an additive for fuels made up of a mass composition of camphor (5-25%), menthol (0.1-10%), ethyl acetate (5-25%), naphthalene (10-35%), ethyl ether (5-25%), acetone (5-25%) and petroleum jelly, and an aromatic hydrocarbon and aliphatic alcohol based solvent to complete its composition. According to the reference, the additive is added to the fuel at 0.01-5% ratio, promoting change in its physical-chemical properties, particularly the increase of octane rating in resulting fuel, to provide more complete combustion with reduction of pollutant gases emission.

In another reference, GB444026A, regarding cars fuels, it is revealed a composition basically formed by a mixture composed of: (1) gasoline; (2) anhydrous ethanol, substantially anhydrous or absolute and (3) by a secondary or tertiary aromatic alcohol represented by formulas (C₁₀H₁₆O), (C₁₀H₁₈O) or (C₁₀H₂₀O). As to aromatic alcohol, according to reference, menthol is a typical example, or, a mixture of previously mentioned alcohols, or a substance containing substantial proportion of aliphatic and acyclic unsaturated primary alcohols, compounds that are similar to those represented by the three previous formulas, and which can be described, for example, by geranial C₁₀H₁₉O, or citronellal C₁₀H₂₀O, or by an essential oil containing a similar alcohol.

Moreover, non-patent reference (Maurice L. Q., Lander H., Edwards T., Harrison III W. E.—Advanced aviation fuels: a look ahead via historical perspective—Fuel (2001) 747-756) describes as possibility for alternative supply to aviation kerosene, the insertion of naphthalenic molecules from heavier cuts of refine processes, especially those from residual coal that show high content of polynuclear asphaltenes, and schist and tar oil. These loads can be co-processed by catalytic cracking and integrated to kerosene cuts obtained by atmospheric distillation. Additional fractions of naphthenic provide a composition for aviation fuels that shows more stability and energetic load, thus assuring higher resistance to oxidation in higher temperatures and avoiding formation of gum in fuel injection into combustion chamber. The possibility of using renewable cycloalkane derivatives in aviation kerosene, which may represent more than 50% of naphthenic molecules, is one of these invention goals. This measure may add qualitative benefits to kerosene, mainly the increase of energy density of resulting fuel, and more reduction in kerosene freezing point, which generate operational advantages like fuel tank smaller volume and best performance of airplanes in high altitudes.

### DISCLOUSURE OF INVENTION

This patent application approaches a new biofuel alternative for use in aviation sector, starting from obtention and production routes of renewable sources compounds that may be used for aviation kerosene composition.

In this sense, naphthenic molecules (cycloalkanes) obtained from renewable sources, as replacement to naphthenic and aromatic molecules from fossil sources, will be used as enrichment or addition components to aviation kerosene.

The process of obtention of naphthenic molecules is based on hydrogenolysis catalytic reactions, from hydro cycloalkanes derivatives substrata, or from their precursor compounds, which are found in biomass, for instance, in essential oils containing menthol, citral, citronellal, and any of their isomers and combinations.

Hydro cycloalkanes derivatives substrata are exemplified here by menthol and isopulegol. The potential candidate to supply fossil naphthenic and aromatic compounds in aviation kerosene are cycloalkane derivatives produced in hydrogenolysis catalytic process, because these compounds show resistance to oxidation, low freezing point and absence of sulfur and nitrogen in their chemical structure.

Hydrogenolyses heterogeneous catalysts properties involve requirements needed to reaction, that is, specific area and good metal dispersion in hydrogenation phase. Heterogeneous catalysts were investigated to assess the best conversion and selectivity conditions related to p-menthane and other cycloalkanes obtention, which are produced during reaction.

Catalysts adequate to p-menthan and cycloalkanes derivatives obtention process are a physical mixture composed of hydrogenating catalysts and acid catalysts, or hydrogenating metals in acid supports (bifunctional catalysts). As to hydrogenation catalysts, that is, supported catalysts of noble metals from groups 6, 7, 8, 9 and 10 of periodic table, the metals referred to are more specifically selected from group corresponding to nickel, platinum, palladium, ruthenium, rhodium, molybdenum, cobalt, tungsten, or mixtures of them. The preferred metal are selected among nickel, platinum or palladium, and platinum is the most preferred. The metallic content in catalyst ranges from 0.01-10%, preferably with 0.01-5% in weight.

Adequate supports to hydrogenolysis catalyst can be any support that show specific area and acidity adequate to hydrogenolysis, especially supports corresponding to metallic oxides like alumina, silica, zirconia, titania or aluminum silicates with zeolitic structure, the most adequate and preferably used corresponding to alumina, silica, silica-alumina, sulfated zirconia, zeolite, Y, acid polymeric resin, for example, Amberlyst15®, zeolite HZSM-5, zeolite faujasite, ferrierite, zeolite beta, clays, natural zeolites and, additionally, active coal.

As to necessary acidity for hydrogenolysis phase of start substrata, that is, hydroxycycloalkanes, adequate acid heterogeneous catalysts are associated to hydrogenolysis catalysts, thus propitiating a physical mixture of catalysts, or otherwise, through catalysts where hydrogenating metallic phase is carried to support with acid characteristics. Any acid heterogenous catalyst or supported metallic catalyst must present acidity and specific area adequate to hydrogenolysis reaction. Acid catalysts adequate to reaction are represented by sulfonated acid polymeric resins, protonated zeolites and sulfated zirconia, clays or natural zeolites. The physical mixture made up or hydrogenation catalyst and acid catalyst shows a mass hydrogenating catalyst/acid catalyst ratio within interval 10:1 to 1:10, preferably within 5:1 to 1:5, and more preferably between 2:1 and 1:2.

In order to establish experimental results uniformity, the hydrogenation catalysts activation or reduction phase was conducted in a reduction unit external to autoclave reactor. Catalytic tests were carried out in series Parr 4560 reactor, with gas feed tank, liquid substratum feed (pressure burette), collectors for liquid and gaseous samples and series Parr PID 4875 controller.

As to processing conditions to obtain p-menthan and cycloalkanes derivatives, the temperature range used is 70-250° C., more preferably between 80 and 220° C., and preferably between 100 and 200° C. Pressure range, in its turn, goes from 1-70 bar, more preferably between 2 and 50 bar, and preferably between 4 and 17 bar. The agitation range is between 100 and 1000 rpm, more preferably between 300 and 800 rpm. Reaction time involving catalytic hydrogenation process varies between 1 and 48 hours, preferably between 2 and 36 hours, more preferably between 4 and 24 hours. Reaction feed conditions were defined according to catalyst mass and liquid substratum volume ration, which varies between 0.0001-0.05 g/ml, more preferably between 0.001-0.02 g/ml. Feed load is a solution in weight of hydroxycycloalkanes substrata, preferably menthos and isopulegol, whose concentration varies between 0.1% and 99.9% (p/v), preferably between 1.0% and 70% (p/v), and more preferably between 5.0% and 40% (p/v), where hydroxycyclealkane compound is diluted in isoparaffinic solvent containing from 5 to 30 carbons, preferably from 9 to 25 carbons in their chain.

Biofuel composition, obtained from catalytic hydrogenation process, is constituted of the mixture resulting from catalytic process that involves p-menthan and cycloalkane derivatives. Biofuel formulation with commercial aviation kerosene (KAV-1), or with aviation gasoline, is a mixture composed of cycloalkanes (obtained in hydroxycycloalkanes substrata hydrogenolysis reaction) and aviation fuel in 1:100 to 100:1 ratio, in volume.

### Reduction of Catalysts

Hydrogenolysis heterogeneous catalysts applied in catalytic conversion reactions, was made of one same methodology of reduction or activation. Especially catalysts, for example, of platinum (Pt) and palladium (Pd), were reduced in a U form glass reactor that supports temperatures of around 500° C. The powder catalyst, previously pulverized and weighted in the desired amount to the reaction was added to a U form glass reactor, to be then fed with reducing gas H₂, with reduction flow of 30 ml/min.

The catalysts reduction thermal condition employed in the invention was conducted with heating rate of 10° C./min, until it reaches final temperature of 350° C., which is kept during the 1-hour reaction period. Catalyst mass cooling up to room temperature, after its reduction, occurred because of the H₂ flow itself, which keeps catalyst material under reducing conditions until the moment of using it in hydrogenolysis reaction as such. The cooled reactor is then disconnected from the furnace with mass content being immediately transferred to Parr reactor and stored in reducing conditions by successive purges with H₂.

The invention concentrations, related to biomass hydrogenolysis process, cycloalkane compounds application thus obtained in aviation kerosene compositions are shown in the following examples:

### EXAMPLE 1

Hydrogenolysis reaction was conducted using menthol with 99% purity content, with a feed load of 6.3% in menthol weight in isoparaffinic solvent C₂₂-C₂₅.

Such experiments were carried out in autoclave reactor, with 100 mL feed in pressure burette of recently prepared solution referred to. Platinum catalysts [Pt(5%)/Al₂O₃] and acid polymeric resin of sulfonated divinylbenzene AMBERLYST 15® (A15), supplied by Rohm & Haas®, were used in form of physical mixture of catalysts. Platinum catalyst previously reduced in the already described reduction conditions, was quickly weighted in analytic scale amounting to 1.0 g mass, and an amount of 0.5 g of A15 resin. Both catalysts were transferred to Parr® reactor, which was readily closed to H₂ purges realization, thus assuring that the air initially contained in reactor dead volume was removed to keep the platinum catalyst under reducing conditions. It was then connected to reactor heating jacket, and when the defined reaction temperature was reached, the feed of menthol 6.3% solution liquid, previously loaded in pressure burette, was then carried out, considering reaction starting point with mechanical agitation kept in rotation of 600 rpm. Along the experimental run, samples were taken in regular intervals of 60 minutes after the first hour of reaction, whose total time was 240 minutes. Final samples of each experiment were analyzed by gaseous chromatography and mass spectrometry (CG-MS). The conversion and selectivity results most representative of the more preferably demanded temperature range are shown in the table below:

Only with hydrogenating catalyst (control reaction)

| | Catalyst | | | Conversion | Selectivity | Selectivity |
|---|---|---|---|---|---|---|
| | Pt(5%)/Al₂O₃ | T | P | (menthol) | (p-menthan) | (neomenthol) |
| Reaction | (g) | (° C.) | (bar) | (%) | (%) | (%) |
| | 1.0 | 190 | 10 | 71 | 26 | 74 |

Physical mixture of catalysts

| | Catalyst | Catalyst | | | Conversion | Selectivity | Selectivity |
|---|---|---|---|---|---|---|---|
| | Pt(5%)/Al₂O₃ | A15 | T | P | (menthol) | (p-menthan) | (cycloalkanes) |
| Reaction | (g) | (g) | (° C.) | (bar) | (%) | (%) | (%) |
| 1 | 1.0 | 0.5 | 80-220 | 5.5 | 98 | 64 | 30 |
| 2 | 1.0 | 0.5 | 80-220 | 10 | 96 | 67 | 30 |

The above results show that with the use of physical mixture of hydrogenating catalysts and acid, conversions and selectivity presented in menthol hydrogenolysis reactions are superior as compared to control reaction (only with hydrogenating catalyst), and the output in cycloalkanes, including p-menthan, in employed reaction conditions, is adequate to industrial application.

### EXAMPLE 2

Another hydrogenolysis reaction was carried out using menthol with 99% purity content, using feed load of 6.3% in menthol weight in isoparaffinic solvent C₂₂-C₂₅. These experiments were conducted in autoclave reactor, by means of 100 mL feed in pressure burette of the recently prepared solution referred to. Platinum catalysts [Pt(5%)/Al₂O₃] and zeolite ZSM-5 in protonic form with silica/alumina ratio (SAR=40) were used to form a physical mixture of catalysts, and to propitiate hydrogenating and acid properties to reaction environment. The previously reduced platinum catalyst, in already described reduction conditions, was quickly weighted in analytic scale amounting a mass of 1.0 g, and an amount of 0.5 g of zeolite HZSM-5. Both catalysts were transferred to Parr® reactor, which was readily closed to H₂ purges realization and to assure that the air initially contained in the reactor dead volume was removed to keep the platinum catalysts under reducing conditions. It was then connected to reactor heating jacket, and when the defined reaction temperature was reached, the liquid feed (6.3% menthol solution) was then carried out, considering reaction starting point with mechanical agitation kept in rotation of 600 rpm. The reaction lasted 240 minutes. Another menthol hydrogenolysis experiment, using bifunctional catalyst Pt(1%)/HZSM-5, was carried out with the same amount of metallic mass in reaction conditions similar to those shown above. Final samples of each experiment were analyzed by CG-MS. The conversion and selectivity results most representative of the more preferably demanded temperature range are shown in the table below:

Physical mixture of catalysts

| | Catalyst | Catalyst | | | Conversion | Selectivity | Selectivity |
|---|---|---|---|---|---|---|---|
| | Pt(5%)/Al₂O₃ | HZSM-5 | T | P | (menthol) | (p-menthan) | (cycloalkanes) |
| Reaction | (g) | (g) | (° C.) | (bar) | (%) | (%) | (%) |
| 1 | 1.0 | 0.5 | 80-220 | 5.5 | 28 | 54 | 38 |
| 2 | 1.0 | 0.5 | 80-220 | 10 | 45 | 50 | 21 |

Bifunctional catalyst

| | | | | | | | Selectivity |
|---|---|---|---|---|---|---|---|
| | Catalyst | | | Conversion | Selectivity | Selectivity | (p-menthan |
| | Pt(1%)/HZSM-5 | T | P | (menthol) | (p-menthan) | (cycloalkanes) | isomers) |
| Reaction | (g) | (° C.) | (bar) | (%) | (%) | (%) | (%) |
| | 5.0 | 80-220 | 10 | 99 | 54 | 27 | 19 |

According to results displayed above, the use of bifunctional catalyst has propitiated improved conversion and selectivity to cycloalkanes, including p-menthan and p-menthan isomers, thus indicating that the amount of acid support mass is decisive to convert practically all menthol in cycloalkanes.

### EXAMPLE 3

A volumetric amount of 70 mL p-menthan with 99.8% purity, cycloalkane representative of menthol hydrogenolysis reaction, described in previous examples, was added to the 630 mL of aviation kerosene KAV-1. The components were then homogenized under magnetic agitation in a beaker, and the content resulting from the mixture is finally transferred to a sampling vial, where fuel tests with the mentioned mixture corresponding to BKAV10 thus obtained were carried out.

Similarly, a sample of BKAV20 was obtained through the mixture of a volume of 126 mL p-menthan with 540 mL of KAV-1, these components were also homogenized, and the resulting content was transferred to a sampling vial for fuel tests.

BKAV10 and BKAV20 samples were tested by a series of essays defined in ANP Norm 137, representative for KAV-1 characterization, particularly tests related to: sulfur content, distillation by ASTM methodology; residue; specific mass; flashpoint; cold filter clogging point; carbon residue; ash content; acidity rate; carbon, hydrogen and nitrogen contents; calorific value and lubrificity.

The table below displays the results of these tests for BKAV10 and BKAV20 mixture compositions, comparing them to pure KAV-1 characterization data, in relation to physical, chemical and combustion characteristics for evaluation of compatibility of naphthenic addition (renewable cycloalkanes, like p-menthan) in commercial aviation kerosene final composition:

| | PURE | | SAMPLE | | SAMPLE | | |
|---|---|---|---|---|---|---|---|
| ESSAYS | KAV-1 | | BKAV10 | | KAV20 | | METHODS |
| Sulfur content (% mm) | 0.306 | | 0.278 | | 0.245 | | EN ISO 20884 |
| Distillation 10% recovered vol. (° C.) | 172.9 | | 173.9 | | 171.9 | | ASTM D-86 |
| Distillation 50% recovered vol. (° C.) | 197.9 | | 193.9 | | 188.9 | | ASTM D-86 |
| Distillation 90% recovered vol. (° C.) | 227.9 | | 228.9 | | 226.9 | | ASTM D-86 |
| Distillation final point temperature | 247.9 | | 247.9 | | 245.9 | | ASTM D-86 |
| Residue (% volume) | 1.1 | | 0.9 | | 1.0 | | ASTM D-86 |
| Loss (%) | 0.4 | | 0.3 | | 1.0 | | ASTM D-86 |
| Specific mass at 20° C. (kg/m³) | 789.9 | | 791.0 | | 792.0 | | ASTM D-4052 |
| Flashpoint Tag (° C.) | 45.0 | | 45.0 | | 47.0 | | ASTM D-56 |
| Cold filter clogging point (° C.) | <−30° C. | | <−30° C. | | <-30° C. | | ASTM D-6371 |
| Ramsbotton carbon residue at final | 0.07 | | 0.07 | 0.01 | n.d. | | ASTM D-524 |
| 10% of distillation (% mm) | | | | | | | |
| Ash content (% mm) | 0.002 | | 0.001 | | 0.001 | | ASTM D-482 |
| Acidity rate (mg KOH/g) | 0.015 | | 0.012 | | 0.012 | | ABNT NBR- |
| | | | | | | | 14448 |
| Water and sediments content | 0 | | 0 | | 0.05 | | ASTM D-2709 |
| (% vol./vol.) | | | | | | | |
| Carbon content (% m/m) | 84.00 | 0.21 | 81.84 | 0.40 | 81.15 | 0.25 | ASTM D-5291 |
| Hydrogen content (% m/m) | 13.44 | 0.20 | 13.51 | 0.23 | 14.02 | 0.03 | ASTM D-5291 |
| Nitrogen content (% m/m) | 0 | | 0.01 | | 0.01 | | ASTM D-5291 |
| Superior calorific value (kcal/kg) | 10854 | 11 | 10830 | 11 | 10897 | 2 | ASTM D-4809 |
| Superior calorific value (kJ/kg) | 45444 | 46 | 45511 | 46 | 45624 | 8 | ASTM D-4809 |
| Inferior calorific value (kcal/kg) | 10339 | 11 | 10352 | 11 | 10353 | 2 | ASTM D-4809 |
| Inferior calorific value (kJ/kg) | 43289 | 46 | 43341 | 46 | 43345 | 8 | ASTM D-4809 |

| | | | | |
|---|---|---|---|---|
| Lubrificity (m) | 585.0 | 665 | 649 | ASTM D-6079 |

(*) NA—not available

1. A biomass hydrogenolysis catalytic process comprising:
a catalytic hydrogenation reaction of hydroxycycloalkanes derivatives to obtain biofuel for aviation, biokerosene composed mainly by cycloalkanes.

2. The process according to claim 1, wherein the catalytic system includes mixtures of hydrogenation heterogeneous catalysts and acid heterogeneous catalysts, or bifunctional catalysts constituted of acid support and active phase by hydrogenating metal.

3. The process according to claim 2, wherein the mass ratio of hydrogenation heterogeneous catalyst:acid heterogeneous catalyst comprises 5:1 to 1:5 range, more preferably the range between 2:1 and 1:2.

4. The process according to claim 2, wherein the hydrogenation heterogeneous catalyst comprises supports among inorganic oxides with metal from groups 6, 7, 8, 9 and 10, or their combinations.

5. The process according to claim 2, wherein the hydrogenation catalysts are supported by metals from groups 6, 7, 8, 9 and 10, selected from Pt, Pd, Ru, Rh, Mo, Co, Ni, W, preferably Pt, Pd and Ni.

6. The process according to claim 2, wherein the hydrogenating active metal content is carried to catalyst support in range 0.01% to 10%, more preferably 0.01% to 5% in weight.

7. The process according to according to claim 2, wherein the acid heterogeneous catalyst is any catalyst with acidity and specific area adequate to the reaction, and it is preferably alumina, silica, silica-alumina, sulfated zircon, coal, zeolite Y, acid polymeric resin Amberlyst 15, zeolite HZSM-5, zeolite faujasita, ferrierite, zeolite beta, clays, natural zeolites and active coal.

8. The process according to claim, wherein the 1 biomass used in cycloalkanes obtention is constituted by menthol, citral, citronellol, citronellal and isopulegol and any of their isomers and combinations of them, including those essential oils derivatives.

9. The process according to claim 1, wherein a reaction substratum is constituted by a feed load that comprises a hydroxycycloalkanes substrata solution chosen from menthol and isopulegol, whose concentration ranges from 1.0% to 99.9% (p/v), where hydroxycyclealkane is diluted in isoparaffinic solvent containing from 5 to 30 carbons in its chain.

10. The process according to claim 1, wherein the hydrogenation reaction is carried or in temperature range of 70 to 250° C., in pressure range of 1 to 70 bar, and agitation range between 100 to 1000 rpm.

11. The process according to claim 10, the reaction time ranging from 1 to 48 hours, preferably from 2 to 36 hours.

12. The process according to the reaction feed conditions are defined according to ratio of catalyst mass per solution volume, ranging from 0.0001 to 0.05 g/mL.

13. A composition involving cycloalkanes obtained through biomass hydrogenolysis catalytic process comprising a mixture composed of cycloalkanes and aviation fuel in ratio 1:100 to 100:1, in volume.

14. The composition according to claim 13, wherein the cycloalkane is mainly pure p-menthan, its isomers and combinations of them.

15. The composition according to claim 13, wherein aviation fuel is a commercial aviation kerosene KAV-1 or Jet-1, and aviation gasoline.

16. A method for using cycloalkanes obtained through the biomass hydrogenolysis catalytic process according to claim 1, the cycloalkanes being use as biofuel, in fuel compositions to in jet turbine propulsion and turbo engine propulsion airplanes and airplanes that use alternative or combustion engines.
